(19) United States
(12) Patent Application Publication
(10) Pub. No.: US 2011/0201785 A1
(43) Pub. Date: Aug. 18, 2011

(54) METHOD FOR OPTIMIZING PROTEINS HAVING THE FOLDING PATTERN OF IMMUNOGLOBULIN

(75) Inventors: Johannes Buchner, Ihrlerstein (DE); Matthias Feige, Memphis, TN (US); Dorothee Ambrosius, Laupheim (DE); Barbara Enenkel, Warthausen (DE)

(73) Assignee: BOEHRINGER INGELHEIM INTERNATIONAL GMBH, Ingelheim am Rhein (DE)

(21) Appl. No.: 13/000,700

(22) PCT Filed: Jun. 30, 2009
(86) PCT No.: PCT/EP09/58225

§ 371 Date: Apr. 21, 2011

(30) Foreign Application Priority Data
Jun. 30, 2008 (DE) 102008030331.3

(51) Int. Cl. C07K 16/00; C07K 14/00

(52) U.S. Cl. 530/387.1; 530/350

(57) ABSTRACT

The invention relates to a method for optimizing the biophysical properties of molecules and derivatives of the Ig superfamily. The method is characterized in that as yet unrecognized helical structural elements with unknown structural, stability and folding roles have been identified as important determinants of correct and efficient structuring of antibody domains. The novel process for positively influencing the antibody properties and properties of other proteins that have the Ig folding pattern now consists of optimizing the properties of the short helical elements and in the transplantation of these elements between Ig domains.

### BACKGROUND TO THE INVENTION

1. Technical Field

The present invention relates to a method for optimising the biophysical properties of proteins of the immunoglobulin (Ig) superfamily. It is thus exceptionally suitable for use on antibodies. However, it is not restricted solely to these, but can theoretically be extended to all the members of the immunoglobulin superfamily, but also to the derivatives thereof, such as e.g. Fc-fusion proteins. The invention thus also relates to methods of preparing proteins of this kind and their medical use.

2. Background

Biomolecules such as proteins, polynucleotides, polysaccharides and the like are increasingly gaining commercial importance as medicines, as diagnostic agents, as additives to foods, detergents and the like, as research reagents and for many other applications. The need for such biomolecules can no longer normally be met—for example in the case of proteins—by isolating molecules from natural sources, but requires the use of biotechnological production methods.

The biotechnological preparation of proteins typically begins with the isolation of the DNA that codes for the desired protein, and the cloning thereof into a suitable expression vector. After transfection of the recombinant expression vector into suitable prokaryotic or eukaryotic expression cells and subsequent selection of transfected, recombinant cells the latter are cultivated in bioreactors and the desired protein is expressed. Then the cells or the culture supernatant is or are harvested and the protein contained therein is worked up and purified.

Antibodies, particularly the subclass immunoglobulin G (IgG), are among the most important proteins produced biopharmaceutically. They have a wide range of applications from basic research through diagnostics to a range of therapies, e.g. the treatment of tumours. Antibodies are complex glycosylated protein molecules, in the case of IgG made up of two light and two heavy chains (see FIG. 1). The recognition and binding of the antigens take place via two identical antigen binding sires, so-called paratopes (see FIG. 1). The target structure of the antibody, the antigen, is not only highly specifically recognised by the latter but its binding is also coupled to a plurality of so-called effector functions which are mediated by the Fc fragment (cf. FIG. 1). The most important effector functions include inter alia the activation of the complement system (complement-dependent cytotoxicity: CDC) and antibody-dependent cell-mediated cytotoxicity (ADCC).

In spite of the range of applications antibodies are not yet used as widely as would be desired, primarily on account of the very high manufacturing costs. Therefore, a variety of strategies have been adopted for improving the molecule and the manufacturing processes. Points of attach for improving the biological properties of an antibody are for example modifications to the affinity and antigen specificity and modulation of the Fc effector functions. Other approaches are directed to reducing the heterogeneity of the molecule, which is caused for example by precursors, hydrolytic breakdown products, enzymatic cleaving of C-terminal amino acid groups of proteins, deaminations, different glycosylation patterns or wrongly linked disulphide bridges, or the improvement of the physicochemical properties of antibodies, such as stability and solubility, for example. Optimising the properties of antibodies thus has a potentially extremely broad range of applications.

Every protein has to undergo a structuring process, known as protein folding, in order to be able to perform its function inherent in the defined final structure. In this multi-stage structuring process which frequently leads via folding intermediates, there may be misfoldings and aggregations. There are a great many diseases that can be attributed to protein misfoldings or are associated with them, as proteins either do not achieve their native folded state or do not remain in this native state. These include, for example, Alzheimer's, Parkinson's and various amyloidoses. If protein misfoldings of this kind occur in biotechnological production processes, this is at the expense of product titre, yield, quality and/or stability.

A number of scientific studies have already dealt with the clarification of the structuring process of antibodies, known as antibody folding (Goto, Y. and Hamaguchi, K., Journal of Molecular Biology 156, 891-910, 1982; Thies, M. J. W. et al., Journal of Molecular Biology 293, 67-79, 1999; Feige, M. J. et al., Journal of Molecular Biology 365, 1232-1244, 2007; Feige, M. J. et al., Journal of Molecular Biology 344, 107-118, 2004). Antibodies belong to the so-called Ig superfamily which is very widespread in nature. Besides the folding studies on antibodies and the fragments thereof, other members of this Ig superfamily have also been thoroughly investigated as to their folding process (Cota, E. et al., Journal of Molecular Biology 305, 1185-1194, 2001; Hamill, S. J. et al., Journal of Molecular Biology 297, 165-178, 2000; Paci, E. at al., Proceedings of the National Academy of Sciences of the United States of America 100, 394-399, 2003). The following picture has emerged of the current state of research: The structuring of the proteins of the Ig superfamily begins around a few hydrophobic amino acids in the core of the pleated sheet structure (especially strand B, C, E and F) and then concludes in the complete structuring starting from this folding core. FIG. 2 shows the typical topology of a member of the Ig superfamily, beta2-microglobulin. Strands B, C, E and F are marked, which as already mentioned are postulated to be the core of the folding process for Ig proteins in general.

### SUMMARY OF THE INVENTION

The present invention relates to a biotechnological process for preparing antibodies or proteins that have the immunoglobulin folding pattern, characterised in that the natural helical elements are optimised. Preferably this optimisation is carried out by introducing additional salt bridges internal to the helix and/or by removing helix breakers or helixdestabilising groups (proline and/or glycine).

In another aspect the invention relates to a biotechnological process for preparing antibodies or proteins which have the immunoglobulin folding pattern, characterised in that the natural or optimised helical elements are transplanted. This transplanting is preferably carried out in domains which have no or few optimal helical elements. In a particularly preferred embodiment, one or more helical elements are transferred from at least one constant domain C_{L}, C_{H}2 and/or C_{H}3 into at least one constant C_{H}1 domain and/or variable domain (e.g. V_{L} or V_{H}).

In another aspect the invention relates to processes for improving the biophysical properties of proteins which have the immunoglobulin folding pattern, characterised in that at least one amino acid in the Ig domain is replaced by another amino acid that increases the likelihood of the formation of a helix, preferably an a helix.

The formation probability is preferably calculated using an algorithm, particularly the AGADIR algorithm. Preferably, the replaced amino acid is located in the region between two β pleated sheet strands, particularly of type A and B and/or E and F. The replaced amino acid may be located in a region that already has a helical structure. The purpose of an amino acid exchange of this kind in an existing helical element is to increase the probability of helix formation of this element. The helix formation can be increased for example if the amino acid to be replaced in the Ig domain is proline or glycine, preferably if it is located at least in the second position (i→i+2) after the preceding n-pleated sheet strand or at most in the penultimate position (i→i−2) before the next n-pleated sheet strand. Proline and glycine are replaced by an amino acid which is neither proline nor glycine, preferably by alanine. Another possibility is the introduction of salt bridges by inserting an amino acid that has a charged side chain in such a way that it is at a spacing (i→i+3), (i→i+4) or (i→i+5) from an amino acid which has a side chain of the opposite charge. At least two amino acids are optionally inserted for this purpose which have side chains with an opposite charge, the spacing between the replaced amino acids being such that the side chains are able to form a salt bridge. In a preferred embodiment, the exchanged amino acids are separated from one another by 2 (i→i+3), 3 (i→i+4) or more amino acids (i→i+5). Amino acids with side chains that are negatively charged under physiological conditions may be glutamic acid or aspartic acid, while arginine, lysine or histidine may have positively charged side chains under these conditions. In a preferred embodiment, the position at which arginine, lysine or histidine is inserted or is possibly already present is closer to the C-terminus than the position where glutamic acid or aspartic acid is inserted or is optionally already present. Thus, a double salt bridge can be inserted in which a sequence is produced wherein 3 amino acids are located in positions i and i+3, i+4 or i+5 as well as i+7, i+8 or i+9, where the amino acids in positions i and i+7, i+8 or i+9 have side chains of the same charge, whereas the amino acid at position i+3, i+4 or i+5 has an opposite charge. For this purpose, 3 corresponding amino acids may be inserted by mutation, possibly even fewer if corresponding amino acids are already present in the starting sequence. In a double salt bridge of this kind, aspartic acid, glutamic acid or arginine is preferably present in the central position i+3, i+4 or i+5. Preferred embodiments are characterised in that after the exchange the protein contains a helical element with the sequence KPKDTLMISR (SEQ ID NO:8), KAEDTLHISR (SEQ ID NO:9), TKDEYERH (SEQ ID NO:10), TPEQWKSHRS (SEQ ID NO:16), and/or SKADYEKHK (SEQ ID NO:11).

In another aspect the present invention relates to the transplantation of suitable helical elements into domains that have no or few optimum helical elements, such as for example the Ig domain of beta2-microglobulin (SEQ ID NO:3), the variable domains (V_{L}, V_{H}) or the constant domain C_{H}1 of immunoglobulins. The transplanted elements may originate for example from the constant immunoglobulin domains C_{L}, C_{H}2 or C_{H}3 or may be variants of such elements, optimised by processes according to the invention. The transplantation is preferably carried out using a method in which 4 to 12 successive amino acids (preferably about 10 amino acids) are replaced by an amino acid sequence of the same or greater length, while the amino acid sequence inserted has a higher helix formation probability than the replaced sequence. In a preferred embodiment, the inserted sequence is a helical element from the region between the 8-pleated sheet strands A and B and/or E and F of a C_{L} or C_{H} domain of an immunoglobulin. Suitable helical elements have for example the sequence KPKDTLMISR (SEQ ID NO:8) from a human C_{H}2 domain (SEQ ID NO:5, SEQ ID NO:14 or SEQ ID NO:15) or the KAEDTLHISR sequence optimised therefrom (SEQ ID NO:9), TKDEYERH (SEQ ID NO:10) from the murine kappa C_{L} domain (SEQ ID NO:1), TPEQWKSHRS (SEQ ID NO:16) from the human C_{L} domain (SEQ ID NO:13) or SKADYEKHK (SEQ ID NO:11) from the human kappa C_{L} domain (SEQ ID NO:12).

In another aspect the present invention relates to a process for preparing a protein that has an immunoglobulin folding pattern, characterised in that a method as hereinbefore described for improving the biophysical properties of proteins which have the immunoglobulin folding pattern is applied to a protein of this kind and the modified protein thus obtained is expressed in a host cell.

In another aspect the present invention relates to a protein that has an immunoglobulin folding pattern, produced by a method according to the invention as hereinbefore described. Preferably it is an antibody, particularly a complete immunoglobulin, containing two light and two heavy chains.

In another aspect the present invention relates to a protein which has an immunoglobulin folding pattern and at least one variable domain (e.g. V_{L} or V_{H}), characterised in that it contains at least one helical element in this variable domain. Preferably, this helical element has the sequence KPKDTLMISR (SEQ ID NO:8), KAEDTLHISR (SEQ ID NO:9), TKDEYERH (SEQ ID NO:10), TPEQWKSHRS (SEQ ID NO:16), or SKADYEKHK (SEQ ID NO:11). In a preferred aspect of the invention, the variable domain has the ability to bind specifically to an antigen.

In another aspect the present invention relates to a protein that has an immunoglobulin folding pattern and at least one constant domain of the type C_{H}2, characterised in that it contains a helical element in this constant domain which has a higher helix formation probability than a helical element of a C_{H}2 domain occurring naturally in humans. In a preferred embodiment, a protein of this kind contains a C_{H}2 domain which contains a helical element with the sequence KAEDTLHISR (SEQ ID NO:9), TKDEYERH (SEQ ID NO:10), TPEQWKSHRS (SEQ ID NO:16) or the sequence SKADYEKHK (SEQ ID NO:11).

In another aspect the present invention relates to a protein which has an immunoglobulin folding pattern and at least one constant domain of type C_{H}1, characterised in that it contains a helical element in this constant domain which has a higher helix formation probability than a helical element of a C_{H}1 domain occurring naturally in humans. In a preferred embodiment, a protein of this kind contains a C_{H}1 domain which contains a helical element with the sequence KPKDTLMISR (SEQ ID NO:8), KAEDTLHISR (SEQ ID NO:9), TKDEYERH (SEQ ID NO:10), TPEQWKSHRS (SEQ ID NO:16) or the sequence SKADYEKHK (SEQ ID NO:11).

In another aspect the present invention relates to a modified β2-microglobulin, which has at least one helical element in an Ig domain, preferably a helical element with the sequence KPKDTLMISR (SEQ ID NO:8), KAEDTLHISR (SEQ ID NO:9), TKDEYERH (SEQ ID NO:10), TPEQWKSHRS (SEQ ID NO:16), or SKADYEKHK (SEQ ID NO:11).

In another aspect the present invention relates to a protein which has an immunoglobulin folding pattern, which comprises at least one helical element in an Ig domain, which has a higher helix formation probability than a helical element which is contained in one of the sequences SEQ ID NO: 1, SEQ ID NO:12 or SEQ ID NO:13 (C_{L} WT) or SEQ ID NO: 5, SEQ ID NO:14 or SEQ ID NO:15 (C_{H}2 WT). In a preferred embodiment, a protein of this kind contains a helical element with the sequence KAEDTLHISR (SEQ ID NO: 9).

In another aspect the present invention relates to a protein as hereinbefore described for medical use.

In another aspect the present invention relates to a biotechnological method of modifying the biophysical properties of antibodies or proteins which have the immunoglobulin folding pattern, characterised in that optimisation of the natural helical elements is carried out.

In another aspect the present invention relates to a biotechnological method of modifying the biophysical properties of antibodies or proteins which have the immunoglobulin folding pattern, characterised in that transplantation of the natural or optimised helical elements is carried out, preferably in domains which have no or few optimum helical elements.

The advantages of the present invention are in a greater folding efficiency and stability, fewer misfoldings and hence, in the final analysis, a higher product yield with at the same time qualitatively higher-value proteins, greater flexibility in the purification process, a slower unfolding rate, particularly under stress conditions, an improvement in solubility and a lower tendency to aggregation of the proteins according to the invention. Thanks to the greater robustness of the manufacturing process, this new method is distinctly superior to the prior art. The present invention can therefore preferably be applied to processes for preparing recombinant antibodies and Fc-fusion proteins. The present invention may however also be applied to other molecules of the immunoglobulin superfamily including fragments and derivatives or fusion proteins thereof that contain domains with homology to immunoglobulin domains.

### DESCRIPTION OF THE FIGURES

FIG. 1: ANTIBODIES OF THE IGG SUBCLASS

The two light chains are light-coloured, the heavy chains are shown darker. The regions responsible for antigen binding (paratopes), the glycosylation of the C_{H}2 domain and the Fc part that mediates the effector functions are labelled.

FIG. 2: BETA2-MICROGLOBULIN AS REPRESENTATIVE OF THE IGSUPERFAMILY

The β-pleated sheet strands B, C, E and F of the human beta2-microglobulin (SEQ ID NO: 3) are labelled.

FIG. 3: IMMUNOGLOBULIN G TOPOLOGY

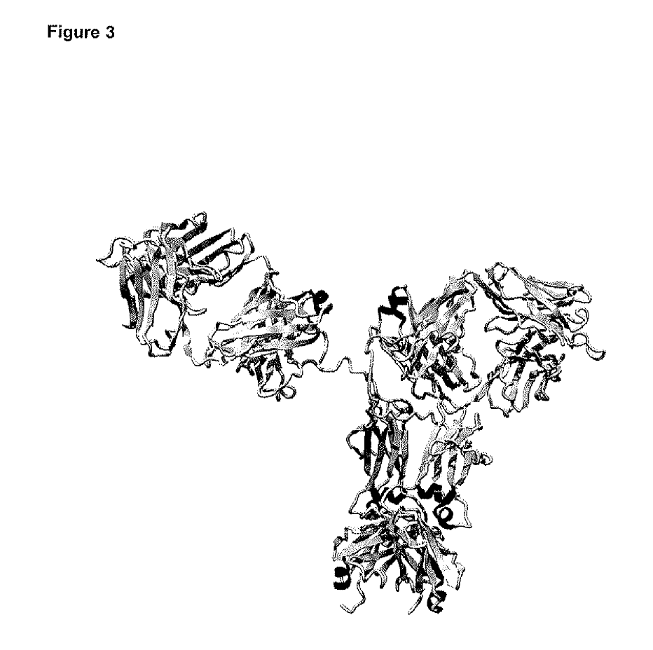

Short helical elements in the Ig topology in the context of an IgG molecule which attach the β-pleated sheets to one another are shown dark.

FIG. 4: LOCATION OF THE HELICAL ELEMENTS IN THE IGG1 C_{L} DOMAIN

In the Figure the location of the helical elements in a constant antibody domain is shown using the example of a human IgG1 C_{L} domain. The β-pleated sheet strands A, B, C, D, E, F and G and the helical elements Helix 1 and Helix 2 are labelled.

FIG. 5: CHARACTERISATION OF THE C_{L}-FOLDING INTERMEDIATE BY NMR SPECTROSCOPY

This Figure shows the peak amplitudes obtained in the first NMR spectrum during refolding for each associated group by comparison with the native peak amplitudes after refolding is complete. The structural elements of the murine kappa C_{L} domain (SEQ ID NO:1) are shown schematically above the peak amplitudes.

FIG. 6: STRUCTURING OF THE IGG C_{L} DOMAIN

The degree of structuring in the folding intermediate of the murine kappa C_{L} domain (SEQ ID NO:1) is determined by NMR spectroscopy. Natively structured regions are shown dark.

FIG. 7: CD-SPECTROSCOPIC EXAMINATION

The CD-spectroscopic examination of the murine kappa C_{L} domain (dashes) (C_{L} WT; SEQ ID NO:1), of the C_{L} domain with the human beta2-microglobulin loops (dashes & dots) (C_{L} to β2m; SEQ ID NO:2) as well as of human beta2-microglobulin (line) (β2m WT; SEQ ID NO: 3) and beta2-microglobulin with the C_{L}-helices (dots) (β2m to C_{L}; SEQ ID NO: 4) is carried out at 20° C. in PBS. C_{L} with the beta2-microglobulin helices (C_{L} to β2m; SEQ ID NO:2) shows the spectrum of an unfolded protein, all the other proteins have the signature of a beta pleated sheet protein.

FIG. 8: INFLUENCE OF THE HELICAL ELEMENTS ON BETA2-MICROGLOBULIN AMYLOID FORMATION

AFM measurements illustrate the reduction in amyloid formation under all conditions with beta2-microglobulin (β2m WT; SEQ ID NO: 3) by transplantation of the C_{L}-helices (β2m to C_{L}; SEQ ID NO: 4). Measurements are carried out at pH 1.5, 3.0 as well as in PBS in the presence and absence of seeds (=fibrils fragmented by ultrasound treatment).

FIG. 9: C_{H}2 DOMAIN OF AN IGG1-MOLECULE

Locating the optimised helix 1 inside the C_{H}2 domain (A) of a human IgG1-molecule (C_{H}2 Helix 1 mutant; SEQ ID NO:6) and the optimisation of helix 1 by inserting additional salt bridges and removing the helix breaker proline (B) (mutation: KPKDTLMISR (SEQ ID NO: 8) to KAEDTLHISR (SEQ ID NO: 9)).

FIG. 10: STRUCTURAL COMPARISON OF THE WILD-TYPE-C_{H}2 DOMAIN WITH THE HELIX1—OPTIMISED MUTANT

FUV-CD spectra (A) and NUV-CD spectra (B), consequently secondary and tertiary structure, are virtually identical for the IgG1 C_{H}2-wild-type domain (dashed line) (C_{H}2 WT; SEQ ID NO: 5) and the Helix1 mutant (solid line) (C_{H}2 Helix1 mutant; SEQ ID NO: 6).

FIG. 11: THERMAL STABILITY INVESTIGATION

The thermal stability of the wild-type C_{H}2 domain (dashed line) (C_{H}2 WT; SEQ ID NO: 5) and of the Helix1 mutant (solid line) (C_{H}2 Helix1 mutant; SEQ ID NO: 6) is measured by FUV-CD spectroscopy at 218 nm. The heating rate is 20° C./h. The melting point of the wild-type is determined as 56.0° C., while that of the mutant is 60.4° C.

### DETAILED DESCRIPTION OF THE INVENTION

Terms and designations used within the scope of this description of the invention have the following meanings defined hereinafter. The general terms “containing” or “contains” include the more specific term “consisting of”. Moreover, the terms “single number” and “plurality” are not used restrictively.

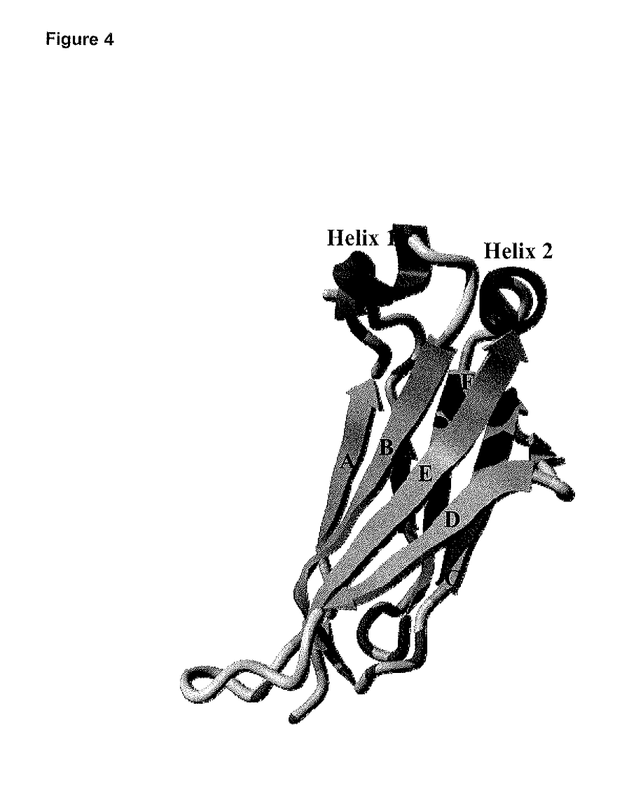

The present invention relates to methods for improving the biophysical properties, particularly for increasing the stability, folding efficiency and for reducing the aggregation of proteins of the immunoglobulin superfamily, as well as the actual proteins thus modified. The immunoglobulin superfamily currently includes more than 760 different proteins. The economically most important group consists of the immunoglobulins (antibodies). There are various categories of immunoglobulins: IgA, IgD, IgE, IgG, IgM, IgY, IgW. Other members are antigen receptors on cell surfaces (e.g. T-cell receptors), co-receptors and costimulatory molecules of the immune system, proteins that are involved in antigen presentation (e.g. MHC molecules), and certain cytokine receptors and intracellular muscle proteins. Proteins of the immunoglobulin superfamily are characterised by common structural elements, the so-called immunoglobulin domains (Ig domains). The Ig domains have a common basic structure. They typically consist of about 70 to 110 amino acids (however, there are also examples with more than 200 amino acids) and frequently contain an intramolecular disulphide bridge. Antibodies of the class IgG for example are made up of four subunits, two identical heavy chains and two identical light chains, in each case, which are joined together by covalent disulphide bridges to form a y-shaped structure. Each light chain contains two Ig domains, a so-called variable (V_{L}) and a constant (C_{L}) Ig domain, while each heavy chain contains four such Ig domains (V_{H}, C_{H}1, C_{H}2, and C_{H}3). Antibodies of classes IgM and IgE contain an additional constant domain (C_{H}4). Ig domains have a characteristic secondary structure, the immunoglobulin folding pattern (in English, “Ig-fold”), a sandwich-like structure with a hydrophobic core which is formed by two sheets of antiparallel n-pleated sheet strands (cf. FIG. 4). The three-dimensional representation is reminiscent of a folded sheet. The peptide groups are located in the sheets and the intervening C-atoms are located in the edges of a multiply folded sheet. The peptide bonds of a plurality of chains interact with one another. The hydrogen bridging bonds needed for stabilisation form along the polypeptide backbone, occurring in pairs at a distance of about 7.0 Å. In the folded sheet, the spacing between adjacent amino acids is much greater than in the significantly more compact a helix. The spacing is 0.35 nm compared with 0.15 nm in the helix.

However, as the side groups are close together, large pleated sheet regions are generally only formed when the side group residues are relatively small and not all equally charged.

To identify β-pleated sheets, CD (“circular dichroism”) spectroscopy and NMR (“nuclear magnetic resonance”) spectroscopy may be used and for statistically evaluating the frequency the Ramachandran Plot may be used (Ramachandran, G. N. et al., J. Mol. Biol. 7, 95-99, 1963). The individual β-strands are referred to as A, B, C, D, E, F, G or C′, C″ etc., according to the order in which they appear in the sequence. The stabilisation of the Ig fold is assisted by interactions of hydrophobic amino acids on the inside of the sandwich, hydrogen bridges between the strands and, if present, a highly conserved disulphide bond between cysteine groups of the B- and F-strands. The number of amino acids located between the two cysteines may vary and is generally between 55 and 75 amino acids. Variable domains of immunoglobulins typically contain 9 β-strands while constant domains typically contain 7β-strands.

The sequence regions between the β-strands are formed by unstructured loops with high sequence variability or, particularly in the constant domains of immunoglobulins, by short helical elements. A helix is a right- or left-handed spiral secondary structure in a protein in which each NH group of the main chain enters into a hydrogen bridging bond with a carbonyl group of the main chain. In the right-handed α-helix, the distance spanned by the hydrogen bridging bond is four amino acids (i+4→i hydrogen bridging bond). In the α-helix one turn corresponds to 3.6 amino acid groups at a level of 1.5 Å (0.15 nm); each amino acid is thus offset by 100°. Further helix shapes are the 3₁₀-helix (i+3→i hydrogen bridging bond) and the π-helix (i+5→hydrogen bridging bond). The side chains of the amino acids are located outside the helix. A typical helix in a protein comprises about 10 amino acids (3 turns or coils), but helical elements made up of only 4 amino acids or helices made up of up to 40 amino acids are also known. Helical secondary structures in proteins can be determined experimentally using methods known per se, for example by x-ray structural analysis or nuclear magnetic resonance spectroscopy (NMR spectroscopy). The helix formation probability may also, however, be determined using suitable algorithms based on the amino acid sequence (Muñoz, V. & Serrano, L. (1997). Development of the Multiple Sequence Approximation within the Agadir Model of α-helix Formation. Comparison with Zimm-Bragg and Lifson-Roig Formalisms. Biopolymers 41, 495-509; Lacroix, E., Viguera A R & Serrano, L. (1998). Elucidating the folding problem of a-helices: Local motifs, longrange electrostatics, ionic strength dependence and prediction of NMR parameters. J. Mol. Biol. 284, 173-191). The AGADIR algorithm described in the abovementioned references is preferred within the scope of the present invention. In the case of constant immunoglobulin domains helical elements are located between the n-pleated sheet strands A and B as well as E and F.

The present invention is based on the finding that helical structures are important for the biophysical properties of proteins which have the immunoglobulin folding pattern. By optimising helical elements of this kind, particularly by changing the amino acid sequence, which bring about an increase in the likelihood of helix formation, preferably of an α-helix, it is possible to improve biophysical properties, and in particular the stability (e.g. thermal stability, pH stability), folding efficiency and solubility can thus be increased and the unfolding speed as well as the tendency to misfolding, aggregation or amyloid formation can be reduced in this way.

Where reference is made hereinafter to “preceding” or “succeeding” positions in amino acid sequences, the word “preceding” means closer to the N-terminus of the sequence, while the term “succeeding” means closer to the C-terminus of the sequence.

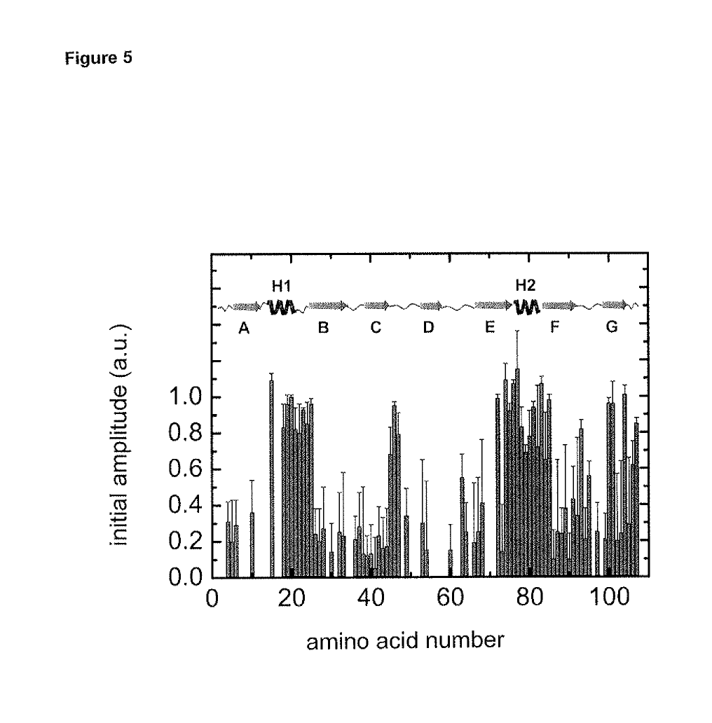

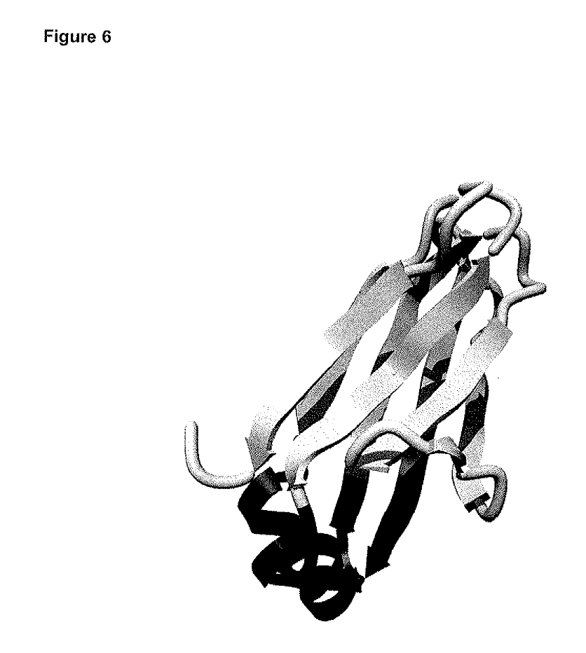

Using high-resolution NMR-spectroscopy the folding path of an antibody domain has been clarified with virtually atomic resolution (cf. FIGS. 5 and 6). This was made possible by the fact that the folding of this domain, the C_{L} domain, is limited by the isomerisation of the Tyr34-Pro35 bond into the native cis state. At low temperatures this process is extremely slow and hence the folding path is directly amenable to NMR-spectroscopy. It was found that en route to the native structure a partially folded structure is formed, a so-called folding intermediate. It is highly significant that in the folding intermediate the short helical elements of the domain are already fully structured, while all the other regions of the protein are only partially structured. FIGS. 5 and 6 illustrate this state of affairs and it is apparent that in particular the short helical elements of the antibody domain are highly structured, whereas the strands B, C, E and F postulated to be the folding nucleus are less structured. By optimising the properties of the helical elements the biophysical properties of antibodies (e.g. stability, solubility, folding efficiency) can be positively influenced, also by transplantation of the helical elements between the domains, for example into the variable domains, which do not have access to the helical elements. Another advantage of the invention is the fact that optimising a protein that essentially has a pleated sheet structure is carried out via short helical elements which are substantially better understood in their properties and are consequently easier to modify than pleated sheet structures.

The present invention relates to a biotechnological method of producing antibodies or proteins which have the immunoglobulin folding pattern, characterised in that the natural helical elements are optimised. Preferably, this optimising is carried out by inserting additional salt bridges internal to the helix and/or removing helix breakers (proline and/or glycine). By a protein that comprises the immunoglobulin folding pattern is meant, within the scope of this invention, a protein which has at least one Ig domain of the structure described hereinbefore. These are In particular members of the immunoglobulin superfamily and therefore preferably immunoglobulins. However, the invention also relates to artificial proteins which do not occur in nature in this form but which have an Ig domain, for example Fc-fusion proteins such as etanercept which is an anti-rheumatoid active substance (TNFR:Fc). By antibodies are meant, in the context of the present invention, not only immunoglobulins, of the kind that occur in nature and may be obtained for example by immunising mammals with an antigen, but also artificial proteins, if they have at least one Ig domain that has a paratope and binds specifically to an antigen, either on its own or together with another Ig domain. Such Ig domains are for example the variable domains of an immunoglobulin (V_{H}, V_{L}.

Of the immunoglobulins that conventionally consist of two light and two heavy chains, those of the class IgG with heavy chains of the subtypes IgG1, IgG2, and IgG4 are preferred. These immunoglobulins may be monoclonal or polyclonal by nature, they may contain primate (particularly human), rodent or other mammalian sequences, and may be chimeric or humanised sequences. Human or humanised immunoglobulins are preferred.

The immunoglobulins may also comprise in their domains, in addition to the optimising processes according to the invention, substitutions, deletions and/or insertions of amino acids which are capable of changing the properties of the molecule. Thus, for example, effector functions such as for example complement-dependent cytotoxicity (CDC), antibody-dependent cellular cytotoxicity (ADCC), apoptosis induction or FcRn-mediated homeostasis may be modulated. By removing potential deamidation, oxidation and glycosylation sites or deleting the C-terminal lysine at the heavy chains, the heterogeneity of the molecule can be reduced, for example.

Besides complete immunoglobulins the skilled man is familiar with a multitude of proteins derived therefrom which contain Ig domains. Thus, he will known for example fragments of immunoglobulins such as Fab, F(ab′)2 or Fc-fragments, Fc-fusion proteins, Fc-Fc-fusion proteins, single-chained antibodies which consist of a fusion of the variable domains of a light and a heavy chain (scFv), single domain antibodies (dAbs) which consist of only the variable domain of a heavy or light chain such as V_{H} V_{HH}, or V_{L} dAbs, including the domain antibodies derived from camelids, as well as minibodies, diabodies, triabodies, and fusion proteins of these constructs.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning or de novo gene synthesis it is also possible to prepare shortened antibody fragments which consist only of the variable regions of the heavy (VH) and light chain (V_{L}). These are known as Fv fragments (fragment variable=fragment of the variable part). As covalent binding via the cysteine groups of the constant chains is not possible in these Fv fragments, these Fv fragments are often stabilised by some other method. For this purpose the variable regions of the heavy and light chains are often joined together by means of a short peptide fragment of about 10 to 30 amino acids, particularly preferably 15 amino acids. This produces a single polypeptide chain in which V_{H} and V_{L} are joined together by a peptide linker. Such antibody fragments are also referred to as single chain Fv fragments (scFv). Examples of scFv antibodies are known and described.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the C_{H}3 region of an IgG or helix structures (“coiled coil structures”) such as the Leucine Zipper domains. In other strategies the interactions between the V_{H} and V_{L} regions of the scFv fragment are used for multimerisation (e.g. dia-, tri- and pentabodies).

The term “diabody” is used in the art to denote a bivalent homodimeric scFv derivative. Shortening the peptide linker in the scFv molecule to 5 to 10 amino acids results in the formation of homodimers by superimposing V_{H}/V_{L} chains. The diabodies may additionally be stabilised by inserted disulphide bridges. Examples of diabodies can be found in the literature.

The term “minibody” is used in the art to denote a bivalent homodimeric scFv derivative. It consists of a fusion protein which contains the C_{H}3 region of an immunoglobulin, preferably IgG, most preferably IgG1, as dimerisation region. This connects the scFv fragments by means of a hinge region, also of IgG, and a linker region.

The term “triabody” is used in the art to denote a trivalent homotrimeric scFv derivative. The direct fusion of V_{H}-V_{L} without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures.

The skilled man is also aware of immunoglobulins from sharks and rays which are known as IgNAR (“new antigen receptor”). These form a dimer of a chain that consists of one variable and five constant regions (Flajnik, M. F., Nature Reviews, Immunology 2, 688-698, 2002).

In addition, the skilled man is also aware of antibodies from llamas or other animals of the camelid family which consist of only two shortened heavy chains each having one variable and two constant domains (Hamers-Casterman, C. et al., Nature 363, 446-448, 1993). The skilled man also knows of derivatives and variants of these camelid antibodies which consist only of one or more variable domains of these shortened heavy chains. Such molecules are also known as domain antibodies. Single domain antibodies are also known based on sequences from other species, e.g. from mice and humans, or in humanised form (Holt et al., Trends in Biotechnology 21(11), 484-490, 2003,). Variants of these domain antibodies include molecules that consist of a plurality of variable domains and are covalently linked to one another by peptide linkers. To prolong the half-life in serum, domain antibodies may also be fused to other polypeptide units, e.g. with the Fc part of immunoglobulins or with a protein occurring in the blood serum, such as albumin, for example.

The terms “helical element” and “helix” are used synonymously in the context of the present invention. They relate to an amino acid sequence of 4 to 12 amino acids, preferably 6 to 12, most preferably 8, 9, or 10 amino acids, which can form a helix.

By “optimising” in the context of the present invention is meant a change in the primary structure of a protein, by which the likelihood of forming a helical element in this protein is increased or by which a helical element is created in this protein, with the objective of improving the biophysical properties of this protein, particularly its folding efficiency, stability, solubility and tendency to aggregation (which is reduced by the optimisation). A preferred method of changing the primary structure of a protein is to mutate its amino acid sequence, i.e. the exchange (substitution), removal (deletion) or introduction (insertion) of at least one amino acid. This is normally done by correspondingly changing the deoxyribonucleic acid (DNA) that codes this amino acid sequence and subsequently expressing this (recombinant) DNA in a host cell. The skilled man has standard methods available to him for doing this.

In another aspect the invention relates to a biotechnological process for preparing antibodies or proteins that have the immunoglobulin folding pattern, characterised in that transplantation of the natural or optimised helical elements is carried out. Preferably this transplantation is carried out into domains that have no or few optimum helical elements. By transplantation is meant, in this context, the replacement of an amino acid sequence of 4 to 12 amino acids by another amino acid sequence of the same length. In a particularly preferred embodiment, one or more helical elements are transferred from at least one constant domain C_{L}, C_{H}2 and/or C_{H}3 into at least one constant C_{H}1 domain and/or variable domain (V_{L} or V_{H}).

In another aspect the invention relates to methods of improving the biophysical properties of proteins, that have the immunoglobulin folding pattern, characterised in that at least one amino acid in the Ig domain is replaced by another amino acid that increases the probability of the formation of a helix. The formation probability is preferably calculated using an algorithm, particularly the AGADIR algorithm. Preferably, the exchanged amino acid is in the region between two β-pleated sheet strands, particularly of type A and B or E and F. The exchanged amino acid may be in a region that already has a helical structure. The objective of an amino acid exchange in an existing helical element is then to increase the helix formation probability of this element. The helix formation can be increased for example if the amino acid to be substituted in the Ig domain is proline or glycine, and preferably if it is located at least in the second position (i→i+2) after the preceding β-pleated sheet strand or at most in the penultimate position (i→i+2) before the next β-pleated sheet strand. Proline or glycine are replaced by an amino acid that is neither proline nor glycine, preferably by alanine. Another possibility is the introduction of salt bridges by introducing an amino acid that has a charged side chain in such a way that it is at a spacing (i→i+3), (i→i+4) or (i→i+5) from an amino acid that has a side chain of the opposite charge. If desired, at least two amino acids are inserted that have side chains of opposite charge, while the spacing between the exchanged amino acids is selected so that the side chains are able to form a salt bridge. In a preferred embodiment the exchanged amino acids are separated from one another by 2 (i→i+3), 3 (i→i+4) or more amino acids. Examples of amino acids with negatively charged side chains under physiological conditions that may be used include glutamic acid or aspartic acid, while arginine, lysine or histidine have positively charged side chains under these conditions. In a preferred embodiment, the position at which arginine, lysine or histidine is inserted or is optionally already present is closer to the C-terminus than the position at which glutamic acid or aspartic acid is inserted or is optionally already present. Also, a double salt bridge can be inserted in which a sequence is produced wherein 3 amino acids are located in positions i and i+3, i+4 or i+5 as well as i+7, i+8 or i+9, where the amino acids in positions i and i+7, i+8 or i+9 have side chains of the same charge, but the amino acid at position i+3, i+4 or i+5 has an opposite charge. For this purpose, 3 corresponding amino acids may be inserted by mutation, possibly even fewer if corresponding amino acids are already present in the starting sequence. In a double salt bridge of this kind, aspartic acid, glutamic acid or arginine is preferably present in the central position i+3, i+4 or i+5. A preferred embodiment is characterised in that after the exchange the protein contains a helical element with the sequence KPKDTLMISR (SEQ ID NO:8) from the human IgG C_{H}2 domain (SEQ ID NO:5, SEQ ID NO: 14 or SEQ ID NO:15) or the helix sequence KAEDTLHISR (SEQ ID NO:9) optimised therefrom, the sequence TKDEYERH (SEQ ID NO:10) from the murine kappa C_{L} domain (SEQ ID NO:1), the sequence TPEQWKSHRS (SEQ ID NO:16) from the human lambda C_{L} domain (SEQ ID NO:13) or the sequence SKADYEKHK (SEQ ID NO:11) from the human kappa C_{L} domain (SEQ ID NO:12).

In another aspect the present invention relates to the transplantation of suitable helical elements into domains that have no or few optimum helical elements, such as for example the Ig domain of beta2-microglobulin (SEQ ID NO:3), the variable domains (V_{L}, V_{H}) or the constant domain C_{H}1 of immunoglobulins. The transplanted elements may originate for example from the constant immunoglobulin domains C_{L}, C_{H}2 or C_{H}3 or may be variants of such elements, optimised by processes according to the invention. The transplantation is preferably carried out using a method in which 4 to 12 successive amino acids (preferably about 10 amino acids) are replaced by an amino acid sequence of the same or greater length, while the amino acid sequence inserted has a higher helix formation probability than the replaced sequence. In a preferred embodiment, the inserted sequence is a helical element from the region between the β-pleated sheet strands A and B and/or E and F of a C_{L} or C_{H} domain of an immunoglobulin. Suitable helical elements have for example the sequence KPKDTLMISR (SEQ ID NO:8) from the human C_{H}2 domain (SEQ ID NO:5, SEQ ID NO:14 or SEQ ID NO:15) or the KAEDTLHISR sequence optimised therefrom (SEQ ID NO:9), the sequence TKDEYERH (SEQ ID NO:10) from the murine kappa C_{L} domain (SEQ ID NO:1), the sequence TPEQWKSHRS (SEQ ID NO:16) from the human C_{L} domain (SEQ ID NO:13) or the sequence SKADYEKHK (SEQ ID NO:11) from the human kappa C_{L} domain (SEQ ID NO:12).

In another aspect the present invention relates to a process for preparing a protein that has an immunoglobulin folding pattern, characterised in that a method as hereinbefore described for improving the biophysical properties of proteins that have the immunoglobulin folding pattern is applied to a protein of this kind, and the modified protein thus obtained is expressed in a host cell. Methods of preparing proteins by the expression of recombinant DNA in host cells and subsequent purification of the desired expressed protein (protein of interest) are sufficiently well known to the skilled man. In particular the skilled man will be familiar with methods of expressing immunoglobulins in eukaryotic host cells, preferably mammalian cells, most preferably cell lines from the ovary of the Chinese hamster (Cricetulus griseus, CHO cells) or cell lines from murine myeloma cells (e.g. NS0 cells). Certain antibody formats such as for example domain antibodies may also advantageously be produced in prokaryotic host cells (e.g. E. coli) or yeast cells.

In another aspect the present invention relates to a protein that has an immunoglobulin folding pattern, produced by a method according to the invention as hereinbefore described. Preferably it is an antibody, particularly a complete immunoglobulin, containing two light and two heavy chains.

In another aspect the present invention relates to a protein that has an immunoglobulin folding pattern and at least one variable domain (V_{L} or V_{H}), characterised in that it contains a helical element in this variable domain. Naturally occurring variable domains do not contain helical elements of this kind and can be improved in their biophysical properties by the introduction of such elements. In one embodiment, a variable domain of this kind contains a helical element with a greater helix formation probability than any amino acid sequence of the same length that occurs naturally in a variable domain of an immunoglobulin. The reference for naturally occurring variable domains of this kind may be the variable domains that are deposited in the data base of the NCBI GenBank under accession numbers AAK19936 (IgG1 VH) and AAK62672 (IgG1 VL). In preferred embodiments, the variable domain according to the invention contains a helical element with the sequence KPKDTLMISR (SEQ ID NO:8), KAEDTLHISR (SEQ ID NO:9), TKDEYERH (SEQ ID NO: 10), TPEQWKSHRS (SEQ ID NO:16) or SKADYEKHK (SEQ ID NO: 11). Particularly preferably, the helical element is located between the pleated sheet strands E and F.

In another aspect the present invention relates to a protein that has an immunoglobulin folding pattern and at least one constant domain of type C_{H}2, characterised in that it contains a helical element in this constant domain that has a higher helix formation probability than a helical element of a C_{H}2 domain occurring naturally in humans. SEQ ID NO: 5 may serve as a reference for such a domain. In a preferred embodiment, a protein of this kind contains a C_{H}2 domain which contains a helical element with the sequence KAEDTLHISR (SEQ ID NO: 9), TKDEYERH (SEQ ID NO:10), the sequence TPEQWKSHRS (SEQ ID NO:16) or SKADYEKHK (SEQ ID NO:11). Preferably, the helical element is located between the pleated sheet strands A and B and/or E and F of the C_{H}2 domain.

In another aspect the present invention relates to a protein that has an immunoglobulin folding pattern and at least one constant domain of type C_{H}1, characterised in that it contains a helical element in this constant domain which has a higher helix formation probability than a helical element or any amino acid sequence of the same length of a C_{H}1 domain occurring naturally in humans. In a preferred embodiment, a protein of this kind contains a C_{H}1 domain which contains a helical element with the sequence KPKDTLMISR (SEQ ID NO:8), KAEDTLHISR (SEQ ID NO:9), TKDEYERH (SEQ ID NO:10), TPEQWKSHRS (SEQ ID NO:16) or the sequence SKADYEKHK (SEQ ID NO:11). Preferably, the helical element is located between the pleated sheet strands A and B and/or E and F of the C_{H}1 domain.

In another aspect the present invention relates to a modified β2-microglobulin which has at least one helical element in an Ig domain, preferably a helical element with the sequence KPKDTLMISR (SEQ ID NO:8), KAEDTLHISR (SEQ ID NO:9), TKDEYERH (SEQ ID NO:10), TPEQWKSHRS (SEQ ID NO:16), or SKADYEKHK (SEQ ID NO:11).

In another aspect the present invention relates to a protein that has an immunoglobulin folding pattern which comprises at least one helical element in an Ig domain that has a higher helix formation probability than a helical element that is contained in one of the sequences SEQ ID NO: 1, SEQ ID NO:12 or SEQ ID NO: 13 (C_{L} WT) or SEQ ID NO: 5, SEQ ID NO: 14 or SEQ ID NO: 15 (C_{H}2 WT). In a preferred embodiment, a protein of this kind contains a helical element with the sequence KAEDTLHISR (SEQ ID NO: 9).

In another aspect the present invention relates to a protein that has an immunoglobulin folding pattern and contains the sequence SEQ ID NO: 4, SEQ ID NO: 6 and/or SEQ ID NO: 9.

In another aspect the present invention relates to a protein as hereinbefore described for medical use in therapy or diagnostics. The medical use of antibodies an other proteins with Ig folding patterns is known to the skilled man and a number of such substances are licensed as drugs (e.g. Rituximab, Trastuzumab, Etanercept). In particular the skilled man is familiar with methods of preparing formulations of such substances (for example physiologically buffered aqueous solutions) and for administering medicaments of this kind when indicated (for example by intravenous injection or infusion).

In another aspect the present invention relates to a biotechnological method of modifying the biophysical properties of antibodies or proteins that have the immunoglobulin folding pattern, characterised in that the natural helical elements are optimised.

In another aspect the present invention relates to a biotechnological method of modifying the biophysical properties of antibodies or proteins that have the immunoglobulin folding pattern, characterised in that transplantation of the natural or optimised helical elements is carried out, preferably in domains that have no helical elements or less suitable helical elements.

The following are further definitions and explanations that are of importance in connection with the present invention:

The proteins of the present invention are preferably produced by recombinant expression in a host cell. An expression vector is used which is introduced into the host cell. The expression vector contains the “gene of interest”, which comprises a nucleotide sequence of any length which codes for a product of interest. The gene product or “product of interest” is generally a protein, polypeptide, peptide or fragment or derivative thereof. However, it may also be RNA or antisense RNA. The gene of interest may be present in its full length, in shortened form, as a fusion gene or as a labelled gene. It may be genomic DNA or preferably cDNA or corresponding fragments or fusions. The gene of interest may be the native gene sequence, or it may be mutated or otherwise modified. Such modifications include codon optimisations for adapting to a particular host cell and humanisation. The gene of interest may, for example, code for a secreted, cytoplasmic, nuclear-located, membrane-bound or cell surface-bound polypeptide.

The term “nucleic acid”, “nucleotide sequence” or “nucleic acid sequence” indicates an oligonucleotide, nucleotides, polynucleotides and fragments thereof as well as DNA or RNA of genomic or synthetic origin which occur as single or double strands and can represent the coding or non-coding strand of a gene. Nucleic acid sequences may be modified using standard techniques such as site-specific mutagenesis, PCR-mediated mutagenesis or de novo synthesis from oligonucleotide seqences.

Proteins/polypeptides with a biopharmaceutical significance in connection with the present invention include for example antibodies or immunoglobulins and other proteins with an immunoglobulin folding pattern, e.g. members of the immunoglobulin superfamily, and the derivatives or fragments thereof. Generally, these are substances that act as agonists or antagonists and/or have therapeutic or diagnostic applications.

The term “polypeptides” or “proteins” is used for amino acid sequences or proteins and refers to polymers of amino acids of any length. This term also includes proteins which have been modified post-translationally by reactions such as glycosylation, phosphorylation, acetylation or protein processing, for example. The structure of the polypeptide may be modified, for example, by substitutions, deletions or insertions of amino acids and fusion with other proteins, such as for example with the Fc part of immunoglobulins, while retaining its biological activity. In addition, the polypeptides may multimerise and form homo- and heteromers.

Expression vectors may theoretically be prepared by conventional methods known in the art. There is also a description of the functional components of a vector, e.g. suitable promoters, enhancers, termination and polyadenylation signals, antibiotic resistance genes, selectable markers, replication starting points and splicing signals. Conventional cloning vectors may be used to produce them, e.g. plasmids, bacteriophages, phagemids, cosmids or viral vectors such as baculovirus, retroviruses, adenoviruses, adenoassociated viruses and herpes simplex virus, as well as synthetic or artificial chromosomes or mini-chromosomes. The eukaryotic expression vectors typically also contain prokaryotic sequences such as, for example, replication origin and antibiotic resistance genes which allow replication and selection of the vector in bacteria. A number of eukaryotic expression vectors which contain multiple cloning sites for the introduction of a polynucleotide sequence are known and some may be obtained commercially from various companies such as Stratagene, La Jolla, Calif., USA; Invitrogen, Carlsbad, Calif., USA; Promega, Madison, Wis., USA or BD Biosciences Clontech, Palo Alto, Calif., USA.

Eukaryotic or prokaryotic host cells are transfected or transformed with suitable expression vectors. Yeast cells and mammalian cells are preferably used as eukaryotic host cells. The former are, in particular, Kluyveromyces, Saccharomyces cerevisiae, Pichia pastoris and Hansenula, while the latter are particularly rodent cells such as e.g. mouse, rat and hamster cell lines. Bacteria, particularly Escherichia coli, Bacillus subtilis, Pseudomonas (P. aeruginosa, P. putida), Streptomyces, Schizosaccharomyces, Lactococcus lactis, Salmonella typhimurium and Agrobacterium tumefaciens are preferably used as prokaryotic host cells, of which Escherichia coli is particularly preferred. The successful transfection or transformation of the corresponding cells with an expression vector according to the invention results in transformed, genetically modified, recombinant or transgenic cells, which are also the subject of the present invention.

Preferred eukaryotic host cells for the purposes of the invention are hamster cells such as BHK21, BHK TK⁻, CHO, CHO-K1, CHO-DUKX, CHO-DUKX B1 and CHO-DG44 cells or derivatives/descendants of these cell lines. Particularly preferred are CHO-DG44, CHO-DUKX, CHO-K1 and BHK21 cells, particularly CHO-DG44 and CHO-DUKX cells. Also suitable are myeloma cells from the mouse, preferably NS0 and Sp2/0 cells and derivatives/descendants of these cell lines. However, derivatives and descendants of these cells, other mammalian cells including but not restricted to cell lines of humans, mice, rats, monkeys, rodents, or eukaryotic cells, including but not restricted to yeast, insect, bird and plant cells, may also be used as host cells for the production of biopharmaceutical proteins.

The transfection of the eukaryotic host cells with a polynucleotide or one of the expression vectors according to the invention is carried out by conventional methods. Suitable methods of transfection include for example liposome-mediated transfection, calcium phosphate coprecipitation, electroporation, polycation- (e.g. DEAE dextran)-mediated transfection, protoplast fusion, microinjection and viral infections.

The transformation of prokaryotic host cells with a polynucleotide or one of the expression vectors according to the invention is carried out using conventional methods. Suitable methods include for example electroporation, chemical treatment of the cells with for example calcium chloride, magnesium chloride, manganese chloride, polyethylene glycol or dimethylsulphoxide, bacteriophage transduction

According to the invention stable transfection is preferably carried out in which the constructs are either integrated into the genome of the host cell or an artificial chromosome/minichromosome, or are episomally contained in stable manner in the host cell. The transfection method which gives the optimum transfection frequency and expression of the heterologous gene in the host cell in question is preferred.

The host cells are preferably established, adapted and cultivated under serum-free conditions, optionally in media which are free from animal proteins/peptides. Examples of commercially obtainable media include Ham's F12 (Sigma, Deisenhofen, Del.), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif., USA), CHO-S-SFMII (Invitrogen), serum-free CHO-Medium (Sigma), protein-free CHO-Medium (Sigma), YM (Sigma), YPD (Invitrogen) and synthetic “prop-out” yeast media (Sigma). Each of these media may optionally be supplemented with various compounds, e.g. hormones and/or other growth factors (e.g. insulin, transferrin, epidermal growth factor, insulin-like growth factor), salts (e.g. sodium chloride, calcium, magnesium, phosphate), buffers (e.g. HEPES), nucleosides (e.g. adenosine, thymidine), glutamine, glucose or other equivalent nutrients, antibiotics and/or trace elements. Although serum-free media are preferred according to the invention, the host cells may also be cultivated using media which have been mixed with a suitable amount of serum.

For the cultivation of prokaryotic host cells there are numerous known media that are also commercially available. Examples include LB, TB, M9, SOC, YT and NZ media (Sigma).

For the selection of genetically modified cells that express one or more selectable marker genes, one or more suitable selecting agents are added to the medium, or suitable “dropout” media are used which lack additives essential to growth, such as for example amino acids or nucleotides.

Gene expression and selection of high-producing host cells:

The term “gene expression” or “expression” relates to the transcription and/or translation of a heterologous gene sequence in a host cell. The expression rate can be generally determined, either on the basis of the quantity of corresponding mRNA which is present in the host cell or on the basis of the quantity of gene product produced which is encoded by the gene of interest. The quantity of mRNA produced by transcription of a selected nucleotide sequence can be determined for example by northern blot hybridisation, ribonuclease-RNA-protection, in situ hybridisation of cellular RNA or by PCR methods (e.g. quantitative PCR). Proteins which are encoded by a selected nucleotide sequence can also be determined by various methods such as, for example, ELISA, protein A HPLC, western blot, radioimmunoassay, immunoprecipitation, detection of the biological activity of the protein, immune staining of the protein followed by FACS analysis or fluorescence microscopy, direct detection of a fluorescent protein by FACS analysis or fluorescence microscopy.

In another aspect the proteins according to the invention are produced in a process in which production cells are multiplied and used to produce the coding gene product of interest. For this, the selected high producing cells are cultivated preferably in a serum-free culture medium and preferably in suspension culture under conditions which allow expression of the gene of interest. The protein/product of interest is preferably obtained from the cell culture medium as a secreted gene product. If the protein is expressed without a secretion signal, however, the gene product may also be isolated from cell lysates. In order to obtain a pure homogeneous product which is substantially free from other recombinant proteins and host cell proteins, conventional purification procedures are carried out. First of all, cells and cell debris are removed from the culture medium or lysate. The desired gene product can then be freed from contaminating soluble proteins, polypeptides and nucleic acids, e.g. by fractionation on immunoaffinity and ion exchange columns, ethanol precipitation, reversed phase HPLC or chromatography on Sephadex, silica or cation exchange resins such as DEAE. Methods which result in the purification of a heterologous protein expressed by recombinant host cells are known to the skilled man and described in the literature.

The invention will now be described by reference to some embodiments by way of example.

### EXAMPLES

### Abbreviations

AFM: atomic force microscopy
β₂m: beta2-microglobulin
bp: base pair
CD: circular dichroism
C_{H}2: second constant domain of a heavy Ig chain
CHO: Chinese Hamster Ovary
C_{L}: constant domain of a light Ig chain
DHFR: dihydrofolate-reductase
E. coli: Escherichia coli
EDTA: ethylenediamine-N,N,N′,N′-tetraacetic acid
ELISA: enzyme-linked immunosorbant assay
FUV: far ultraviolet
GdmCl: guanidine hydrochloride
GSH: glutathione
GSSG: glutathione disulphide
HSQC: heteronuclear single quantum coherence
HC: heavy chain
HT: hypoxanthine/thymidine
Ig: immunoglobulin
IgG: immunoglobulin G
kb: kilobase
LC: light chain
mAk: monoclonal antibody
MD: molecular dynamics
MTX: methotrexate
NMR: nuclear magnetic resonance
NPT: neomycin-phosphotransferase
NUV: near ultraviolet
PCR: polymerase chain reaction
SEAP: secreted alkaline phosphatase
WT: wild-type

### Methods

### Protein Production in Bacteria and Purification

For the expression of the proteins, the recombinant E. coli bacteria BL21 DE3 (Stratagene, Calif., USA) are cultivated overnight in selective LB medium at 37° C. and 300 rpm in shaking flasks. In order to produce isotope-labelled proteins for NMR measurements, the recombinant bacteria are cultivated in M9 Minimal medium (Sigma) with ¹⁵N ammonium chloride as the sole nitrogen source or optionally additionally ¹³C glucose as the sole carbon source.

Then the “inclusion bodies” are isolated. For this, the bacteria are removed by centrifuging and resuspended in 100 mM Tris/HCl, pH 7.5, 10 mM EDTA, 100 mM NaCl, protease inhibitor. The cells are lysed in a French Press, mixed with 2% v/v Triton X-100 and stirred for 30 min at 4° C. By centrifugation (20,000 rpm, 30 min) the “inclusion bodies” are isolated as a pellet and then resuspended twice in 100 mM Tris/HCl, pH 7.5, 10 mM EDTA, 100 mM NaCl, protease inhibitor and centrifuged off again (20,000 rpm, 30 min). For the proteins β₂m WT (SEQ ID NO: 3), β₂m to C_{L} (SEQ ID NO:4), C_{H}2 WT (SEQ ID NO: 5) and C_{H}2 Helix1-mutant (SEQ ID NO: 6) the inclusion body pellet is then resuspended in 100 mM Tris/HCl, pH 8.0, 10 mM EDTA, 8 M urea and applied to a Q-Sepharose column that had been equilibrated in 100 mM Tris/HCl, pH 8.0, 10 mM EDTA, 5 M urea. All the proteins are in the flowthrough and are refolded overnight in 250 mM Tris/HCl, pH 8.0, 100 mM arginine, 10 mM EDTA, 1 mM GSSG, 0.5 mM GSH at 4° C. by dialysis. Then the protein is concentrated and finally purified through a Superdex75 μg gel filtration column equilibrated in PBS.

For purifying the proteins C_{L} WT (SEQ ID NO: 1), C_{L} P35A (SEQ ID NO: 7) and C_{L} to β₂m (SEQ ID NO:2) which contain an N-terminal His tag, the inclusion bodies are solubilised in 100 mM sodium phosphate (pH 7.5), 6 M GdmCl, 20 mM β-mercaptoethanol for two hours at 20° C. Insoluble components are then eliminated by centrifugation (48000 g, 25 min, 20° C.). The supernatant is diluted five times in 50 mM sodium phosphate (pH 7.5), 4 M GdmCl and applied to a nickel chelate column (Ni-NTA, Qiagen). After washing with five column volumes elution is carried out with 50 mM sodium phosphate (pH 4), 4 M GdmCl. Refolding by dialysis is carried out in 250 mM Tris/HCl, pH 8.0, 5 mM EDTA, 1 mM oxidised glutathione at 4° C. overnight. Aggregates are eliminated by centrifuging (48000 g, 25 min, 4° C.). To remove the N-terminal His tag, 0.25 units of thrombin (Novagen) are added for 16 hours at 4° C. per milligram of protein. After further centrifugation, the proteins are finally purified through a Superdex75 μg gel filtration column equilibrated in 20 mM sodium phosphate (pH 7.5), 100 mM NaCl, 1 mM EDTA.

### CD Spectroscopy

CD measurements are carried out in a Jasco J-715 spectropolarimeter. Measurements are carried out at 20° C. in PBS.

Far UV CD spectra are measured from 195-250 nm at a protein concentration of 50 μM in a 0.2 mm quartz dish, near UV CD spectra are measured from 250-320 nm at a protein concentration of 50-100 μM in 5 mm quartz dishes. Measurements are carried out at 20° C. in PBS. Spectra are accumulated 16-fold in each case, averaged and buffer-corrected. Temperature transitions are measured at 218 nm (C_{H}2 WT/mutant) or 205 nm (C_{L}, β₂m WT/mutants) respectively, in PBS at 10 μM protein concentration in a 1 mm quartz dish at a heating rate of 20° C./h.

### AFM Measurements

For fibrillisation experiments a 100 μM protein solution in PBS 1:1 is mixed with buffer A (25 mM sodium acetate, 25 mM sodium phosphate, pH 1.5 or 2.5). The final pH value is thus at pH 1.5 or pH 3.0, respectively. The solution is incubated for 7 days with gentle tilting at 37° C., then 20 μL of the solution are applied to fresh mica surfaces, washed three times with sterile filtered water and then analysed in the AFM. The AFM contact mode with a scan speed of 1.5 μm/minute is used. Measurements are carried out using a Digital Instruments Multimode Scanning Probe microscope and DNP-S20 tips. “Seeds” are generated from beta2-microglobulin fibrils (pH 1.5) by incubating for 10 minutes in the ultrasound bath. For “seeding” experiments, 2 μl of “seeds” are added to 100 μL of mixture.

### NMR Measurements

Unless stated otherwise, all the spectra are measured at 25° C. in Bruker DMX600, DMX750 and AVANCE900 spectrometers. Assignments are undertaken using standard triple resonance spectra. For refolding experiments and real-time HSQC measurements, C_{L} is unfolded in 2 M guadinium chloride and then diluted 1:10 with ice-cold PBS. The HSQC measurements during the folding process are carried out at 2° C. every 14 min and analysed using SPARKY.

### Gene Synthesis

The sequence region for the wild-type C_{H}2 domain and C_{L} domain is amplified by PCR from a human IgG1-antibody gene or the kappa chain of the murine antibody MAK33 (Augustine, J. G. et al., J. Biol. Chem. 276 (5), 3287-3294, 2001). The P35A mutation is inserted into the C_{L} domain by PCR mutagenesis using mutagenic primers. For expression in E. coli the sequence regions for the C_{H}2 domain of the helix-optimised C_{H}2 mutant, β₂m-WT and the β₂m mutant with the transplanted C_{L}-helix is synthesised de novo (www.geneart.com). For the expression of the complete antibody in CHO-DG44 cells the helix mutations are inserted into the wild-type C_{H}2 domain of an IgG1 antibody gene by PCR mutagenesis using mutagenic primers.

### Eukaryotic Cell Culture and Transfection

The cells CHO-DG44/dhfr^{4-/-} are permanently cultivated as suspension cells in serum-free CHO-S-SFMII medium supplemented with hypoxanthine and thymidine (HT) (Invitrogen GmbH, Karlsruhe, Del.) in cell culture flasks at 37° C. in a damp atmosphere and 5% CO₂. The cell counts and viability are determined with a Cedex (Innovatis) and the cells are then seeded in a concentration of 1-3×10⁵/mL and passaged every 2-3 days.

For the transfection of CHO-DG44, Lipofectamine Plus Reagent (Invitrogen) is used. For each transfection batch a total of 1.0-1.1 μg plasmid-DNA, 4 μL Lipofectamine and 6 μL Plus reagent are mixed according to the manufacturers' instructions and added in a volume of 200 μL to 6×10⁵ cells in 0.8 ml of HT-supplemented CHO-S-SFMII medium. After three hours' incubation at 37° C. in a cell incubator 2 mL of HT-supplemented CHO-S-SFMII medium are added. After a cultivation period of 48 hours the transfection mixtures are either harvested (transient transfection) or subjected to selection. As one expression vector contains a DHFR selection marker and the other one contains an NPT selection marker, 2 days after transfection the co-transfected cells are transferred into CHO-S-SFMII medium without added hypoxanthine and thymidine for the DHFR- and NPT-based selection and G418 (Invitrogen) is also added to the medium in a concentration of 400 μg/mL.

A DHFR-based gene amplification of the integrated heterologous genes is carried out by the addition of the selection agent MTX (Sigma) in a concentration of 5-2000 nM to an HT-free CHO-S-SFMII medium.

### Expression Vectors

For the expression in CHO-DG44, eukaryotic expression vectors are used which are based on the pAD-CMV vector (Werner, R. G. et al., Arzneimittel-Forschung/Drug Research 48, 870-880, 1998) and mediate the expression of a heterologous gene via the combination of CMV enhancer/CMV promoter. The first vector pBI-26 contains the dhfr minigene which acts as an amplifiable selectable marker. In the second vector pBI-49 the dhfr-minigene is replaced by an NPT gene. For this purpose the NPT selection marker, including SV40 early promoter and TK-polyadenylation signal, was isolated from the commercial plasmid pBK-CMV (Stratagene, La Jolla, Calif., USA) as a 1640 by Bsu361 fragment. After a reaction of topping up the fragment ends with Klenow DNA polymerase the fragment was ligated with the 3750 bp Bsu361/Stul fragment of the first vector, which was also treated with Klenow DNA polymerase. Then the NPT gene was modified. It is the NPT variant F240I (Phe240IIe), the cloning of which is described in WO2004/050884.

For the expression in Escherichia coli BL21 DE3 (Stratagene, Calif., USA) the vector pET28a (Novagen) is used.

### Elisa (Enzyme-Linked Immunosorbant Assay)

The quantification of the expressed antibodies in the supernatants of stably transfected CHO-DG44 cells is carried out using ELISA according to standard procedures, using on the one hand a goat anti human IgG Fc fragment (Dianova, Hamburg, Del.) and on the other hand an AP-conjugated goat anti human kappa light chain antibody (Sigma). The standard used is purified antibody of the same isotype as the expressed antibodies in each case.

### SEAP Assay

The SEAP titre in culture supernatants from transiently transfected CHO-DG44 cells is quantified using the SEAP Reporter Gene Assays according to the manufacturer's operating instructions (Roche Diagnostics GmbH).

### ThermoFluor® Method

In order to analyse the thermal stability of the optimised proteins/immunoglobulins, a qPCR system (Mx3005P™; Stratagene) is used, based on the ThermoFluor® method. A solvatochromic/environment-sensitive fluorescent dye is used as an indicator of minor changes in the thermal stability of proteins. This fluorescent dye, which has a small quantum yield in aqueous solution, interacts with hydrophobic, non-native structures of the protein that is unfolding as a result of a temperature rise. The interaction of the dye with protein domains that have already unfolded results in a significant increase in the fluorescence detected (Cummings M. D. et al., Journal of Biomolecular Screening 854-863, 2006).

The measurement of the protein probes in a temperature range of 25° C. to 95° C. at intervals of 1° C. per minute takes place in a volume of 20 μL, while 2 μM protein and 4× SyproOrange (prepared from a 5000× SyproOrange stock solution; Invitrogen) are used in the buffer that is to be tested in each case.

### Example 1

### Procedure for Improving the Biophysical Properties of Immunoglobulin Domains

The first step is to identify the helical elements or the corresponding loops, if no helices are present, in the immunoglobulin domain that has been selected as the target for optimisation. In the case of constant antibody domains, the helices are always located, for example, between the β-pleated sheet strand A and B and E and F (FIG. 4). After identification of these regions, optimisation is carried out according to the following plan:
1. All the proline and/or glycine groups are replaced by another amino acid, preferably alanine (if there is no conflict with point 2 that is to be prioritised). The substitution is only carried out if the group that is to be replaced is not the first group after the preceding β-pleated sheet strand or the last group before the succeeding β-pleated sheet strand.
2. Then the helices are stabilised by the insertion of additional salt bridges. This is done by replacing previously existing amino acids with amino acids having charged side chains of a different charge. Any combination of arginine, lysine, histidine, aspartate or glutamate may be used. The groups that are to be replaced must be separated by two, three or four amino acids, to ensure the formation of the salt bridge in the helix, so that the charged groups inserted will for example have the numbering i and i+3, i and i+4 or i and i+5. All permutations of the above-mentioned groups are possible. Preferably, however, arginine, lysine or histidine is inserted closer to the C-terminus than aspartate or glutamate. Double salt bridges are also theoretically possible, if they accord with the helix length and all the other points specified, for example group i and i+3, i+4 or i+5 and i+7, i+8 or i+9 are replaced as described previously. Groups i and i+7, i+8 or i+9 each have the same charge while the group i+3, i+4 or i+5 has the opposite charge. At position i+3, i+4 or i+5, aspartate, glutamate or arginine should preferably be used. In this step, only solvent-exposed groups located on the surface of the protein are to be replaced.
3. All the remaining groups that do not come under the optimisations described in point 1. and/or 2. are replaced by amino acids which lead to an increase in the probability of formation of an α-helix. The computer algorithm Agadir (Internet address: http://www.embl.de/Services/serrano/agadir/agadir-start.html) or any other algorithm for predicting the probability of formation of an α-helix forms the basis. In this step, only solvent-exposed groups located on the surface of the protein are to be replaced.

For variable antibody domains and/or other antibody domains and/or other immunoglobulin domains in which there are generally no helical elements to be found, the corresponding loops should first be replaced by a helix from a constant antibody domain, preferably the C_{L} domains of the IgG1 subclass of the same organism from which the molecules that are to be optimised originate. Then the optimisation is carried out according to the above procedure.

### Example 2

### Investigating the Protein Folding of the C_{L} Domain

In order to ascertain important determinants of the folding path of an antibody domain, high-resolution structural investigations are carried out on the murine MAK33 C_{L} domain and a spot mutant (C_{L}-P35A). The proteins C_{L} WT (SEQ ID NO:1) and C_{L}-P135A (SEQ ID NO:7) are recombinantly produced in E. coli. The first four N-terminal amino acids in C_{L} WT each result from the chosen cloning strategy into the expression vector pET28a and do not occur naturally in the C_{L} domain. NMR spectroscopy can be used to monitor the folding of the C_{L} domain, after unfolding in the denaturing agent GdmCl, in real time at low temperatures (FIGS. 5 and 6). It is found that the two short helical elements between strands A and B and between strands E and F are already completely structured in the main folding intermediate (FIGS. 5 and 6). It can thus be postulated that they play an important part in the folding process of these and other antibody domains. The speed-determining step of folding the C_{L} domain, before which the folding intermediate is populated, is the isomerisation of the proline group 35 from trans to cis. Therefore, this group is exchanged for alanine, which should always be present in trans. In this way the folding intermediate can be stabilised in equilibrium. NMR investigations on it confirm the kinetic investigations on the WT-C_{L} domain: The two short helices are the only completely structured elements in the C_{L} domain.

### Example 3

### Transplantation of Helical Elements

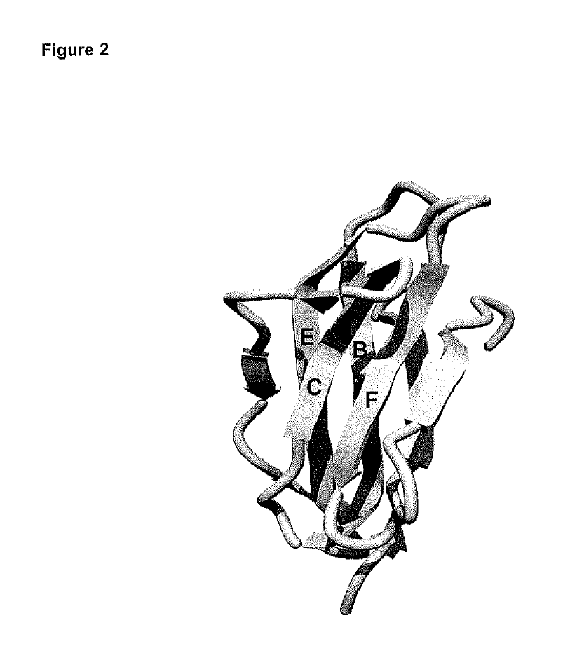

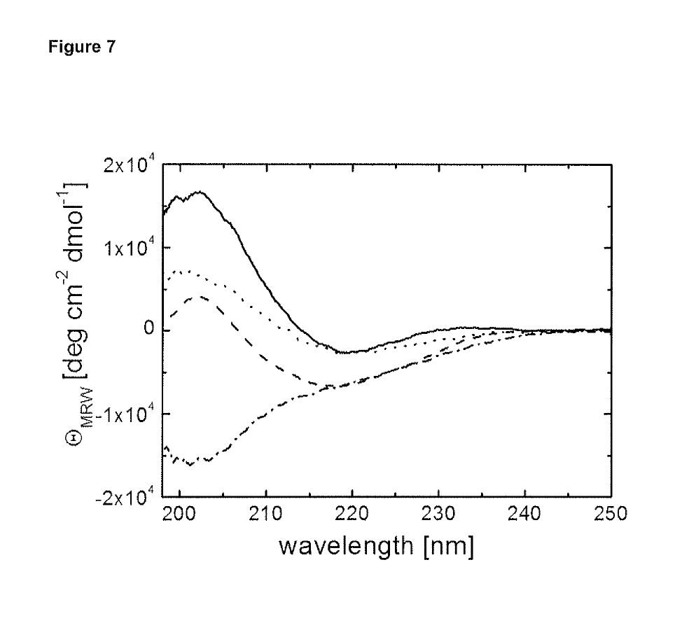

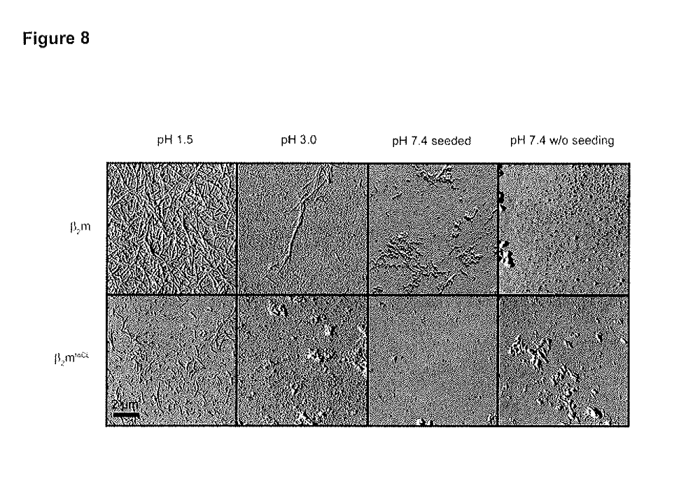

A transfer of the helical elements, especially from the constant domains C_{L}, C_{H}2 and C_{H}3 into the C_{H}1 domain (which has only slightly marked helices) and the variable domains (which have no helices) of an antibody is one possible approach. For additional or alternative optimisation of the helices it is possible for example to resort to additional salt bridges within the helix and to eliminate helix breakers (proline groups or glycine groups). The viability of this approach can be demonstrated by studies on the C_{L} domain of the light kappa chain of a murine IgG and beta2-microglobulin. By genetic modification, the two helical elements in C_{L} (FIG. 5) which connect the β-pleated sheet strands A and B or E and F are exchanged for the corresponding unstructured regions from beta2-microglobulin (FIG. 2) (C_{L} to β₂m; SEQ ID NO: 2). Conversely, the unstructured regions in beta2-microgobulin are replaced by the corresponding helical elements from C_{L} (β₂m to C_{L}; SEQ ID NO: 4). The proteins C_{L} to β₂m and β₂m to C_{L} and, as a control, the wild-type sequences β₂m (SEQ ID NO: 3) and C_{L} (SEQ ID NO: 1) are recombinantly produced in E. coli. The first four N-terminal amino acids in C_{L} WT or the first N-terminal amino acid in C_{L} to β₂m result in each case from the chosen cloning strategy into the expression vector pET28a and do not occur naturally in the C_{L} domain. It can be shown by CD spectroscopy that C_{L} can no longer fold into its native structure in the absence of its helices (=C_{L} to β₂m), whereas beta2-microglobulin becomes significantly less prone to aggregation when the C_{L}-helices are transplanted into the beta2-microglobulin sequence (β₂m to C_{L}) (see FIGS. 7 and 8). Moreover, molecular dynamic simulations show that even in the context of the beta2-microglobulin protein the C_{L}-helices structure themselves and thus in reality constitute robust folding elements. These measurements by way of example are able to show both an essential role for the structuring of antibody domains and a positive influence on the Ig topology.

### Example 4

### Optimisation of the Human IgG1 C_{H}2 Domain

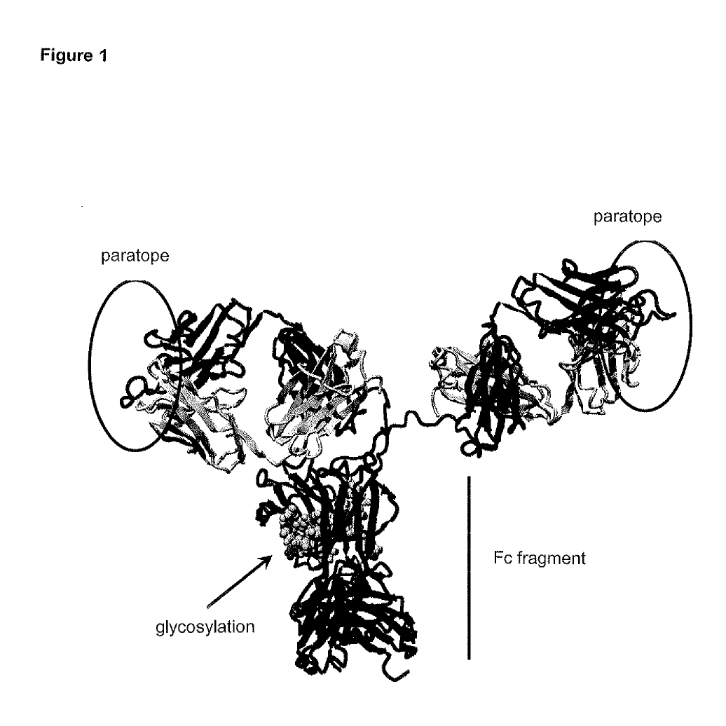

Within the IgG Fc-fragment (FIG. 1) the C_{H}2 domain is the weakest link in terms of stability. In addition, the Fc fragment can be regarded as a general platform of IgG antibodies, so that optimisation of the biophysical properties of the C_{H}2 domain on the one hand should increase the overall stability of the Fc fragment and on the other hand should constitute an optimisation that is universally applicable.

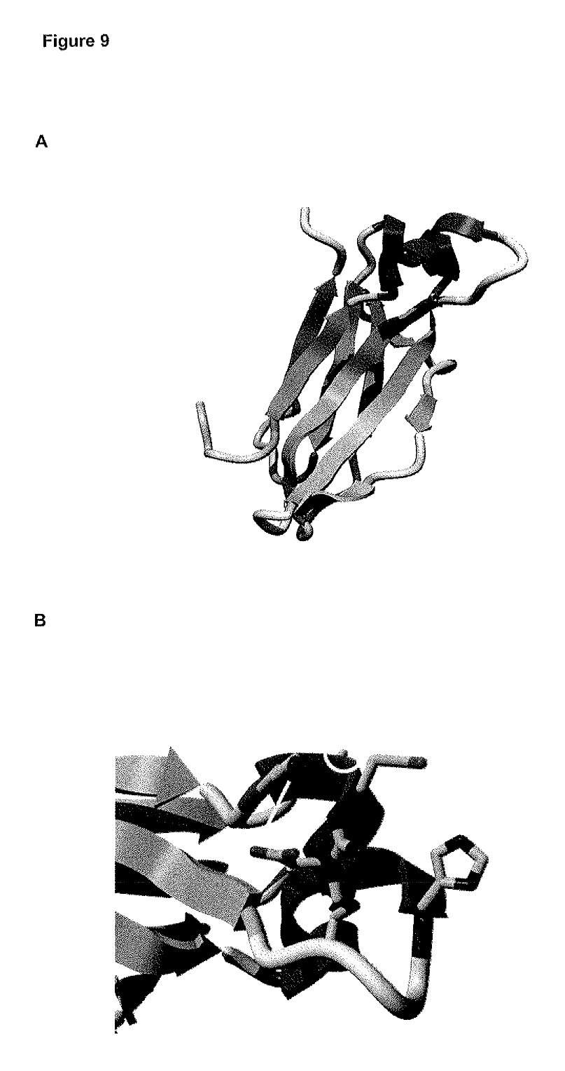

For this example, the first helix of a human IgG1 C_{H}2 domain (FIG. 9A) is selected for optimisation. Additional salt bridges are inserted into it by targeted mutagenesis (FIG. 9B). Both C_{H}2 domains, wild-type (C_{H}2 WT; SEQ ID NO: 5) and Helix1-mutant (C_{H}2 Helix1 mutant; SEQ ID NO: 6), are expressed in E. coli. The first N-terminal amino acid in the C_{H}2 Helix1 mutant results from the chosen cloning strategy into the expression vector pET28a and does not occur naturally in the C_{H}2 domain.

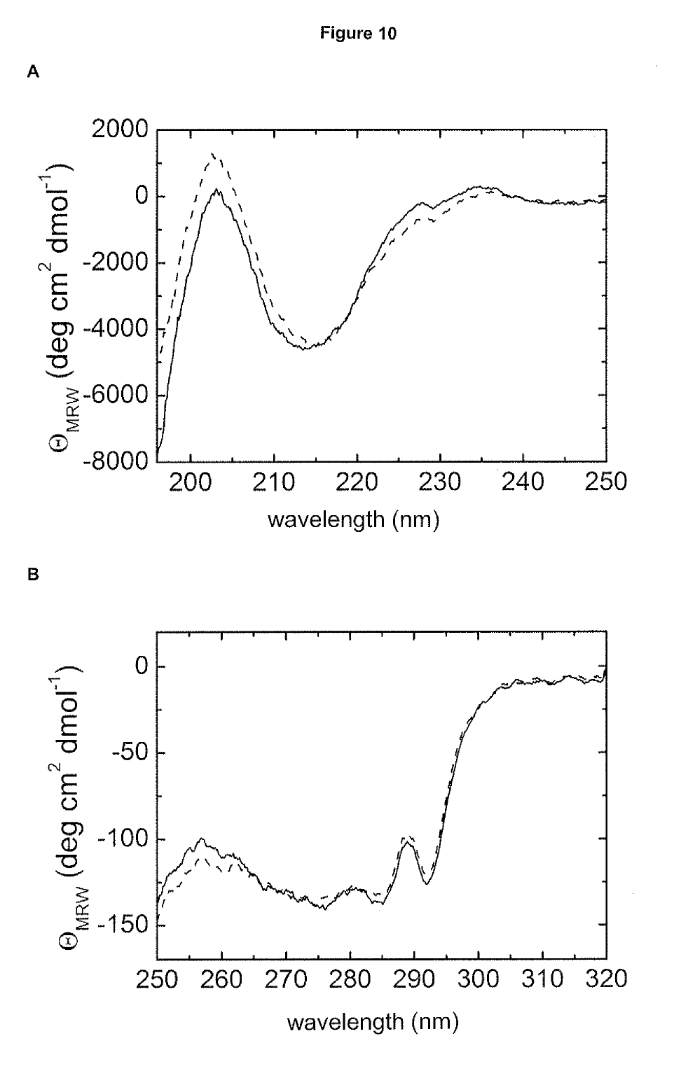

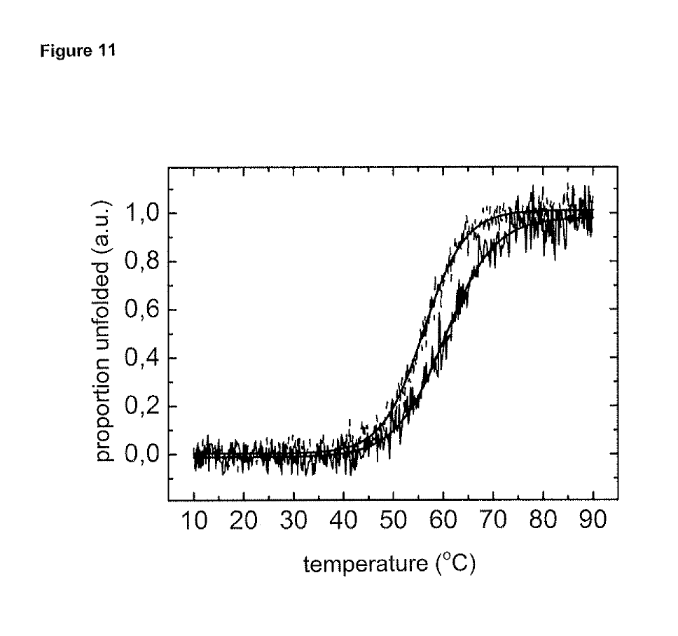

Analyses carried out with the purified proteins show that using this approach it is possible to generate a C_{H}2 domain which is virtually unchanged from the wild-type domain in terms of the secondary structure and tertiary structure (FIG. 10), but has a melting point that is 4-5° C. higher (FIG. 11). In addition, by optimising the first helix, a higher yield can be obtained in the refolding of the recombinant C_{H}2 domain, which is directly indicative of an optimised folding property of this mutated domain.

### Example 5

### Expression of Optimised Antibodies in Cho Cells

By transient transfection of CHO-DG44-cells a check is made first of all to see whether the substitution of the helical sequence element KPKDTLMISR (SEQ ID NO: 8) in the C_{H}2 domain of an IgG1 antibody gene by the helix-optimised sequence element KAEDTLHISR (SEQ ID NO: 9) has an influence on the expression of the IgG1 molecule. Co-transfection is carried out with the following plasmid combinations:
a) control plasmids pBI-26/IgG1-HC and pBI-49/IgG1-LC, which for a monoclonal IgG1-antibody with the sequence region KPKDTLMISR (SEQ ID NO: 8) in the C_{H}2 domain (=wild-type configuration; hereinafter referred to as IgG1-WT)
b) pBI-26/IgG1-HChelix1 and pBI-49/IgG1-LC, which for a monoclonal IgG1-antibody, in which the first helix in the human C_{H}2 domain is optimised by substitution of the sequence region KPKDTLMISR (SEQ ID NO:8) by KAEDTLHISR (SEQ ID NO:9)

3 Pools are transfected for each combination, with equimolar amounts of the two plasmids being used in each co-transfection. After 48 h cultivation harvesting is carried out and the IgG1 titre in the cell culture supernatant is determined by ELISA. Differences in the transfection efficiency are corrected by co-transfection with a SEAP expression plasmid (addition of 100 ng of plasmid DNA to each transfection mixture) and subsequent measurement of the SEAP activity. In all, 2 independent transfection series are carried out. It can be shown that the mutations in the helix region of the C_{H}2 domain the IgG1-molecule do not have an adverse effect on the expression of the antibody. The amounts of product obtained are comparable with those of IgG1 wild-type transfected cells.

For stable transfection of CHO-DG44-cells, co-transfection is carried out with the same plasmid combinations as described above. The selection of stably transfected cells takes place two days after the transfection in HT-free medium with the addition of 400 μg/mL of G418. After the selection, a DHFR-based gene amplification is induced by the addition of 100 nM MTX. For the material production the cells are grown in a 10-day fed-batch process in shaking flasks. The purification is identical for the WT or Helix1-mutant of the antibody. The protein A affinity chromatography (MabSelect rProteinA, GE Healthcare) is carried out according to the manufacturer's instructions, using phosphate buffer (20 mM sodium phosphate, 140 mM sodium chloride, pH 7.5, conductivity 16.5 mS/cm) for the equilibration and 50 mM acetate pH 3.3 for the elution. The eluate is adjusted to a pH of 5.5 by the addition of 1 M Tris pH 8. The purification profiles for the two antibody variants are comparable.

The thermal stability of the antibodies is determined by the ThermoFluor® method. By optimising the natural helical element in the C_{H}2 domain the thermal stability of the Helix1-mutant of the IgG1 antibody can be increased compared with the IgG1-WT antibody under both basic and acidic buffer conditions. In PBS at pH 7.1 the C_{H}2 domain of the Helix1-mutant exhibits a melting temperature that is 8° C. higher than the C_{H}2 domain of the IgG1-WT. Also, in 100 mM acetate pH 3.4, it is even 18° C. higher. This significant increase in the thermal stability of the immunoglobulins is of immense advantage for the biopharmaceutical preparation of therapeutic proteins. Optimisation of the natural helical element of the C_{H}2 domain leads to a significantly improved robustness of the biotechnologically produced therapeutic proteins by replacing the naturally occurring sequence region KPKDTLMISR (SEQ ID NO:8) (this sequence region also occurs for example in the C_{H}2 domains of human IgG2 (SEQ ID NO:14) and IgG4 (SEQ ID NO:15)) with KAEDTLHISR (SEQ ID NO:9). The increased temperature- and pH-stability is particularly advantageous in the process step of virus inactivation in order to increase the product safety of therapeutic proteins, as this step is carried out at an acid pH. Other advantages are the greater flexibility in chromatography and in the protein formulation, the lower tendency to aggregation and the improved storage stability.

1. A biotechnological process for preparing antibodies or proteins that have an immunoglobulin folding pattern and helical elements, characterized in that the natural helical elements are optimized.

2. The process according to claim 1, characterized in that the optimization is carried out by introducing additional salt bridges internal to the helix and/or by removing helix breakers.

3. A biotechnological process for preparing antibodies or proteins that have an immunoglobulin folding pattern and helical elements, characterized in that the natural or optimized helical elements are transplanted.

4. Process according to claim 3, characterized in that one or more helical elements are transferred from at least one constant domain C_{L}, C_{H}2 and/or C_{H}3 into at least one constant C_{H}1 domain and/or variable domain.

5. A process for improving the biophysical properties of proteins that have an immunoglobulin folding pattern, characterized in that at least one amino acid in the Ig domain is replaced by another amino acid that increases the likelihood of the formation of a helix.

6. The process according to claim 5, characterized in that the formation probability is calculated using an algorithm.

7. The process according to claim 5, characterized in that the replaced amino acid is located in the region between two β-pleated sheet strands.

8. The process according to claim 7, characterized in that the replaced amino acid(s) is (are) located in the region between two β-pleated sheet strands of type A and B and/or between two β-pleated sheet strands of type E and F.

9. (canceled)

10. The process according to claim 5, characterized in that proline or glycine is replaced by an amino acid which is neither proline nor glycine.

11. The process according to claim 5, characterized in that an amino acid that has a charged side chain is inserted in such a way that it is at a spacing (i→i+3), (i→i+4) or (i→i+5) from an amino acid which has a side chain of the opposite charge.

12. The process according to claim 11, characterized in that at least two amino acids are inserted which have side chains with an opposite charge, the spacing between the two amino acids being such that the side chains are able to form a salt bridge.

13. The process according to claim 12, characterized in that the two replaced amino acids are separated from one another by 2 or more amino acids ((i→i+3), (i→i+4) or (i→i+5)).

14. The process according to claim 12, characterized in that one of the two inserted amino acids is glutamic acid or aspartic acid, and the other amino acid is arginine, lysine or histidine.

15. The process according to claim 11, characterized in that the position at which arginine, lysine or histidine is inserted or is possibly already present is closer to the C-terminus than the position where glutamic acid or aspartic acid is inserted or is optionally already present.

16. The process according to claim 11, characterized in that a sequence is produced wherein up to 3 amino acids are inserted at positions i and i+3, i+4 or i+5 as well as i+7, i+8 or i+9, while the amino acids and positions i and i+7, i+8 or i+9 have side chains of the same charge, whereas the amino acids at position i+3, i+4 or i+5 have an opposite charge.

17. The process according to claim 16, characterized in that at the central position i+3, i+4 or i+5 aspartic acid, glutamic acid or arginine is introduced or is optionally already present.

18. The process according to claim 1, characterized in that after the exchange the protein contains a helical element with the sequence KPKDTLMISR (SEQ ID NO:8), KAEDTLHISR (SEQ ID NO:9), TKDEYERH (SEQ ID NO:10), SKADYEKHK (SEQ ID NO:11), and/or TPEQWKSHRS (SEQ ID NO:16).

19. The process according to claim 1, characterized in that 4 to 12 successive amino acids are replaced by an amino acid sequence of the same or greater length, while the amino acid sequence inserted has a higher helix formation probability than the replaced sequence.

20. The process according to claim 19, characterized in that the inserted sequence is a helical element from the constant domain of a light (C_{L}) or heavy (C_{H}) immunoglobulin chain.

21-35. (canceled)


SEQUENCE LISTING (16 sequences)

SEQ ID NO: 1 - protein, 107 aa, Mus musculus
  CL WT
GSHMAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 2 - protein, 100 aa, Artificial Sequence
  Description of Artificial Sequence Synthetic polypeptide
MAAPTVSIFPRHPAENGKSASVVCFLNNFYPKDINVKWKIDGSERQNGVLNSWTDQDSKDSTYSMSSTLTLTPTEKNSYTCEATHKTSTSPIVKSFNRNE

SEQ ID NO: 3 - protein, 100 aa, Homo sapiens [sequence omitted]

SEQ ID NO: 4 - protein, 107 aa, Artificial Sequence
  Description of Artificial Sequence Synthetic polypeptide
MGIQRTPKIQVYSRPPSSEQLTSGGNFLNCYVSGFHPSDIEVDLLKNGERIEKVEHSDLSFSKDWSFYLLYYTEFTKDEYERHDEYACRVNHVTLSQPKIVKWDRDM

SEQ ID NO: 5 - protein, 108 aa, Homo sapiens
  IgG1 CH2
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 109 aa, Artificial Sequence
  Description of Artificial Sequence Synthetic polypeptide
MELLGGPSVFLFPPKAEDTLHISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 7 - protein, 107 aa, Artificial Sequence
  Description of Artificial Sequence Synthetic polypeptide
GSHMAAPTVSIFPPSSEQLTSGGASVVCFLNNFYAKDINVKWKIDGSERQNGVLNSWTDQDSKDSTYSMSSTLTLTKDEYERHNSYTCEATHKTSTSPIVKSFNRNE

SEQ ID NO: 8 - protein, 10 aa, Homo sapiens
  Helix motif from IgG1 CH2
KPKDTLMISR

SEQ ID NO: 9 - protein, 10 aa, Artificial Sequence
  Description of Artificial Sequence Synthetic peptide
KAEDTLHISR

SEQ ID NO: 10 - protein, 8 aa, Mus musculus
  Helix motif from the murine kappa light chain CL
TKDEYERH

SEQ ID NO: 11 - protein, 9 aa, Homo sapiens
  Helix motif from the human CL domain of the kappa light chain
SKADYEKHK

SEQ ID NO: 12 - protein, 106 aa, Homo sapiens
  Human CL domain of the kappa light chain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 105 aa, Homo sapiens
  Human CL domain of the lambda light chain
QPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 14 - protein, 109 aa, Homo sapiens
  Human CH2 domain of the heavy IgG2 chain
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 15 - protein, 110 aa, Homo sapiens
  Human CH2 domain of the heavy IgG4 chain
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 16 - protein, 10 aa, Homo sapiens
  Helix motif of the human lambda light chain CL
TPEQWKSHRS